(19) United States
(12) Patent Application Publication
(10) Pub. No.: US 2016/0024592 A1
(43) Pub. Date: Jan. 28, 2016

(54) SINGLE-CELL ANALYSIS AS A SENSITIVE AND SPECIFIC METHOD FOR EARLY PROSTATE CANCER DETECTION

(71) Applicant: THE BOARD OF REGENTS OF THE UNIVERSITY OF TEXAS SYSTEM, Austin, TX (US)

(72) Inventors: Tim Hui-Ming HUANG, San Antonio, TX (US); Chun-Liang CHEN, San Antonio, TX (US); Joseph LIU, San Antonio, TX (US); Chiou-Miin WANG, San Antonio, TX (US); Ian M. THOMPSON, San Antonio, TX (US); Chun-Lin LIN, San Antonio, TX (US)

(73) Assignee: THE BOARD OF REGENTS OF THE UNIVERSITY OF TEXAS SYSTEM, Austin, TX (US)

(21) Appl. No.: 14/775,997

(22) PCT Filed: Mar. 14, 2014
(86) PCT No.: PCT/US14/27346

§ 371 Date: Sep. 14, 2015

Related U.S. Application Data
(60) Provisional application No. 61/784,885, filed on Mar. 14, 2013.

(51) Int. Cl. C12Q 1/68; G01N 33/574

(52) U.S. Cl. CPC C12Q 1/6886; G01N 33/57434; C12Q 2600/158

(57) ABSTRACT

Certain embodiments are directed to methods of measuring single cell levels of biomarkers associated with prostate cancer.

### STATEMENT REGARDING PRIORITY

This Application claims priority to U.S. Provisional Patent Application No. 61/784,885 filed Mar. 14, 2014, which is incorporated herein by reference in its entirety.

### STATEMENT REGARDING FEDERALLY FUNDED RESEARCH

This invention was made with government support under CA113001 awarded by the National Institutes of Health. The government has certain rights in the invention.

### BACKGROUND

Prostate cancer is the second leading cause of cancer related death for men in USA. Based on rates between 2007 and 2009, 16.2% of men will be diagnosed with prostate cancer during their lifetime. The cost of prostate cancer care was $11.85 billion in 2010. In order to improve the survival rate and alleviate the medical burden, sensitive and specific methods for early detection and effective therapeutics are needed.

The current diagnosis of prostate cancer relies primarily on increased prostate specific antigen (PSA) in the blood and abnormal digital rectal examination (DRE). These two methods have limits on sensitivity and specificity for the detection of prostate cancer. The sensitivity for PSA and DRE as a screening test for prostate cancer was 72% and 53% and the specificity was 93% and 84%, respectively. Positive predictive value was 32% for PSA and 21% for digital rectal examination. Thus, approximately four men with elevated PSA levels undergo prostate biopsies to find one with cancer, and some cancerous men with “normal” PSA levels escape detection using PSA/DRE methods.

Thus, there remains a need for additional methods for detecting prostate cancer with increased sensitivity and specificity as compared to PSA and DRE methods.

### SUMMARY

The proof-of-principle study described herein provides a conceptual advance for deciphering inter-clonal heterogeneity of a tumor. Presently, expression profiles of microdissected tissue are commonly used to stratify cancer subtypes (Tamura et al., Cancer Res 67, 5117-25 (2007)). This kind of analysis is conducted under the assumption that uniform gene expression is present in a cell population. Nevertheless, clonal heterogeneity is increasingly detected in primary tumors (Meacham and Morrison, Nature 501, 328-337 (2013)), and novel approaches are needed to analyze gene expression complexity for risk assessment. The reductionist approach described herein has led to the establishment of a binary code system for single-cell analysis. Interestingly, this binary behavior could not be observed when prostate tumors were analyzed in aggregate in a TCGA cohort. While three of six genes identified using the described methods—TRGBR2, GATA3, and CDKN1C, are known tumor suppressors, their up-regulation has also been reported in advanced cancers (Levy and Hill, Cytokine Growth Factor Rev 17, 41-58 (2006)). Irrespective of their tumorigenic roles, these genes display dichotomous expression patterns that can readily be used for clonal analysis of single cells. Genes whose complex expression patterns can be reduced to numeric codes for disease diagnosis can be selected from the pool of known biomarkers or potential biomarkers. In certain aspects new biomarkers may also be identified using the methods described herein. While biomarker expression alterations may not directly contribute to a disease process per se, the genes represent a new class of single-cell binary biomarkers. Thus, the “liquid biopsy” or DIGITAL BIOPSY™ described here have broad applications for detecting rare disease cells isolated from bodily fluids, including blood, saliva, breast milk, and vaginal secretions, and washes or leftover materials from biopsy needles and surgical blades.

Certain embodiments are directed to methods for assessing and/or detecting a disease or condition by single cell analysis. The single-cell approach described herein reduces the possibility of false positives and false negatives. To that end, the methods would assist in early detection of disease or condition (e.g., prostate cancer), improve human health, and decrease unnecessary medical expenses. The invention utilizes much less invasive methods, for example urine samples can be collected post-DRE. In certain aspects the methods describe herein can be used in combination with known methods of detection or diagnosis, for example in prostate cancer screening the methods can be used with other prostate cancer screening methods such as PSA levels in the blood.

The methods described herein are less invasive and use body fluids into which target cells are shed. The target cell can be a diseased or pathogenic cell such as a cancer cell. In certain aspects the body fluid can be blood, cerebrospinal fluid (CSF), saliva, urine, semen, etc. In certain aspects body fluid samples are collected after a procedure that may increase shedding of a target cell into the body fluid. In a further example urine samples can be collected post-DRE. In combination with PSA in the blood or as a stand alone diagnostic, single cell analysis using post-DRE urine samples can be used for detecting prostate cancer. A sufficient number of prostate cells are found in urine, particularly after DRE, for conducting single cell analysis. In certain embodiments the biological sample need not be a fluid sample, but can be a solid sample that is subsequently dispersed, e.g, a biopsy or fecal sample. In certain aspects the biological sample can be a biopsy or other tissue sample. A tissue sample can be treated with various enzymes that degrade extracellular components and free individual cells from the tissue for analysis.

Single-cell analysis can be used to assess and/or measure biomarkers associated with a disease or pathological condition. In certain aspect cell type specific markers can be used to identify a target cell in a sample. Cell type specific markers are those surface proteins that are selectively expressed by a tissue or cell type, e.g., prostate cell, colon cell, liver cell, heart cell, lung cell, etc. A number of such markers are known. In a further aspect disease or pathology related biomarkers can be used to characterize a particular cell. In the example provided here, prostate cells found in a urine sample are analyzed. In certain aspects a urine sample is collected from a patient. In a further aspect the patient had undergone DRE within the last 1, 2, 3, 4, 5, 6, 7, 8, 9, 10, 11, 12, 13, 14, 15, 16, 17, 18, 19, 20, 21, 22, 23, or 24 hours. In other methods a non-urine body fluid is collected or a tissue sample is dispersed for single cell analysis.

Certain embodiments include methods for single cell analysis. Single-cell analysis profiles can provide greater sensitivity and specificity than traditional methods, allowing earlier and more reliable diagnosis of a disease or condition, e.g., prostate cancer. In certain aspects cells are fixed and/or stabilized upon collection and/or isolation. In a further aspect single cells are sorted or selected. Single cells can be sorted manually or by automated sorting or selection. In certain aspects single cells are sorted using a DEPArray or similar technique/instrument. In other aspects single cells are sorted manually by manipulation with a micromanipulator. In certain aspects a target cell is identified by a cell type specific marker(s). A cell type specific marker can include, but is not limited to, one or more of PSA, PSMA, EpCAM, CK7, or CK8. In certain aspects the cell type specific marker is measured or detected and the level and/or presence/absence of biomarkers is determined. In certain aspects a cell type can be identified by which proteins it expresses or does not express. For example a particular marker can be expressed for a number of cell types being derived from a common precursor and specific cell types can then be identified using one or more second markers to further classify the general cell type. In certain aspects analysis of urine can be done in conjunction with method for identifying a particular cell type. Once the particular cell type is identified and isolated other biomarkers can be assessed to characterize each isolated cell. A number of isolated cells are analyzed to obtain a population of characterized cells. In certain aspect the character of the population of characterized cells can be used to determine the diagnosis and/or prognosis of a subject. In a further aspect such a method can be used for assessing cell type character in the blood and detecting and characterizing circulating target cells, such as tumor cells, as a diagnostic or prognostic cancers or metastatic cancers.

Certain embodiments are directed to monitoring a subject over time. Biological samples can be obtain 1, 2, 3, 4, 5, 6, 7, 8, 9, 10, or more times over 1, 2, 3, 4, 5, 6, 7, 8, 9, 10 or more days, weeks, months, or years. For example urine, blood, or other body fluids as well as tissue samples can be obtained over time. The method can be used to monitor patients that are at risk for disease development, such as prostate cancer development or progression. Patients can include patients undergoing therapy/surgery and post-therapy/surgery. A subject can be at risk for disease development based on family history, genomic marker of predisposition, and/or physiologic symptoms that indicate a risk for disease development.

Certain embodiments are directed to methods of detecting prostate cancer cells comprising (a) measuring levels of a biomarker in a single prostate cell isolated from post-digital rectal examination (DRE) urine of subjects; and (b) comparing the single cell levels of the biomarker to a reference to classify the prostate cell as cancerous or non-cancerous. In certain aspects a prostate cell is selected by using a tissue specific marker. A prostate specific marker can be prostate specific antigen (PSA) and/or prostate specific membrane antigen (PSMA). In a further aspect a prostate specific marker is EpCAM and/or CK7/8. In still other aspects the prostate specific marker is PSA, EpCAM, and CK7/8. In certain aspects a biomarker is CXCL6, TGFBR2, GSK3B, CDKN1C, GATA3 and EIF4EBP1. In certain aspects a single prostate cancer cell is isolated using a dielectrophoresis cage array, a microfluidic device, or micromanipulation.

Certain embodiments are directed to methods for detecting prostate cancer cells in a urine sample comprising: (a) concentrating cells in a urine sample; (b) contacting the concentrated cells with a detectable antibody that binds a prostate cell specific marker; and (c) conducting biomarker profiling on the prostate cell. In certain aspects the cell specific marker is prostate specific antigen (PSA) or prostate specific membrane antigen (PSMA). In further aspects the prostate specific marker is EpCAM and/or CK7/8. In still further aspects the cell specific marker is PSA, EpCAM, and CK7/8.

Other embodiments are directed to methods for expressing complex gene expression patterns as binary code strings comprising: identifying and ordering a plurality biomarkers that individually or in combination correlate with a pathological state into a binary code string that is correlated with a diagnosis or prognosis, wherein the biomarkers are genes that exhibit bimodal expression. In certain aspects the biomarkers comprise the genes CXCL6, TGFBR2, GSK3B, CDKN1C, GATA3 and EIF4EBP1. In certain aspects the binary code strings are composed of a 0 representing low expression or 1 representing high expression for each gene.

Certain embodiments are directed to a computer implemented method comprising the steps of (a) obtaining single cell protein level measurements of one or more biomarker, (b) transforming the obtained measurements to a score or ratio, and (c) determining if the measurements indicate the presence of prostate cancer.

Other embodiments include methods of treating a patient having prostate cancer comprising: administering a treatment for prostate cancer to a patient having elevated single cell levels of one or more biomarker.

Certain embodiments are directed to methods of monitoring a subject comprising: (a) measuring levels of a biomarker in a single prostate cell isolated from post-digital rectal examination (DRE) urine of subjects periodically; and (b) comparing the single cell levels of the biomarker to a reference to classify the prostate cell as cancerous or non-cancerous over time. In certain aspects the subject has prostate cancer, is at risk of developing prostate cancer, or is undergoing prostate cancer treatment.

Further embodiments are directed to methods for determining a biomarker profile of a population of representative cells isolated from urine comprising: (a) contacting cells isolated from urine with a detection agent that identifies a population of representative cells in the sample; (b) isolating the identified cells as single cell isolates; (c) conducting biomarker analysis on the each of the isolated single cells to determine a biomarker profile.

Embodiments include methods for determining a biomarker expression profile for detecting and evaluating prostate cancer in a patient comprising: (a) contacting cells isolated from urine obtained from a patient suspected of having prostate cancer with a detection agent that identifies a population of prostate cells in the sample; (b) isolating the identified prostate cells as single cell isolates; (c) conducting prostate cancer biomarker analysis on the each of the isolated single cells to determine a biomarker profile; (d) assessing the biomarker profiles of a plurality of prostate cells and providing an assessment of the patient relating to a diagnosis of prostate cancer or a prognosis for prostate cancer.

Further embodiments include methods for display of a biomarker expression profile comprising: (a) obtaining single cell biomarker profiles for a plurality of target cells isolated from a sample; (b) grouping the single cell biomarker profiles into two or more pathological stages or states based on correlation of the single cell biomarker profile with a normal, benign, or pathological condition; and (c) displaying geometric shapes representing various biomarker profiles, wherein the geometric shape has a size that is proportional to number of cells having a particular profile and an indicator of which state the single cell biomarker profile correlates. In certain aspects the geometric shape is a circle with the radius or diameter of the circle being proportional to the number of binary clones or codes identified in a cell population. In certain aspects the cell with the most developed pathological character can be represented by a red color with a normal cell type being represented by a more subdued color such as green or a pale shade of blue, etc.

The term “isolated” can refer to a cell, nucleic acid, or polypeptide that has had some or substantially all of the non-cellular material (e.g., other components of a biological fluid, extracellular matrix, tissue scaffold, etc.), cellular material, bacterial material, viral material, or culture medium (when produced by recombinant DNA techniques) of their source of origin.

Moieties of the invention, such as oligonucleotides, polypeptides, peptides, antigens, or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.”

The phrase “specifically binds” or “specifically immunoreactive” to a target refers to a binding reaction that is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1C. Urine sample analysis using DEPArray after immunostaining shows heterogeneous PSA expression profiles of cells from urine samples of prostate cancer patients. (A) A flow chart illustration shows the urine sample analysis protocol. (B) Immunostaining of EpCAM, CK7/8, and PSA on representative PSAlow and PSAhigh cells from a urine sample. (C) Bar graphs display PSA levels of triple-positive (EpCAM-CK7/8-PSA) cells from urine samples of two prostate cancer patients and one normal individual.

FIG. 2. Scatter plots show expression profiles of PSA and EpCAM on cells from urine samples of one prostate cancer patient and one BPH patient using DEPArray.

FIG. 3. PSA/PSMA expression patterns in urine samples.

FIG. 4. Diagram of computer implemented aspects of the invention.

FIGS. 5A-5D. Gene expression analysis of urinary prostate cells. (a), Exfoliated prostate cells isolated from urine sediment were positively identified by fluorescent markers PSA (red dye) and PSMA (green dye) for single-cell isolation. (b), Representative examples (#25 and N02) of microfluidic PCR analysis of KLK3 and UBB genes. Ct (threshold cycle) value was the outcome of RT-PCR analysis for fold changes of gene expression. ΔRN: Normalized Reporter=fluorescence intensity of reporter dye divided by that of reference dye. (c), Expression profiles of PPAP2A in 1220 single cells (red hair lines) isolated from normal controls (Ctrl) and patients with benign prostate hyperplasia (BPH), high-grade prostatic intraepithelial neoplasia (HGPIN), and prostate cancer (PCa). Additional single-cell expression profiles are presented in (d), Dichotomous single-cell expression profiles of six genes. Lower panel: A violin graph combining a box plot with a kernel density plot displays a bimodal expression pattern, 0 for low and 1 for high expression, for a given gene in a total of 1220 cells analyzed. Normalized expression values range from 0 to 35.

FIGS. 6A-6B. Parallel coordinate plot analysis of urinary prostate cells. (a), single-cell expression patterns of genes are connected in a string (brown) for a given cell. A total of 1220 connected lines are shown here. A patina line traces an expression path of a cell across two (upper) or three genes (lower). Connectivity paths are converted to binary code-strings with 0 for low and 1 for high expression, respectively. (b), Examples of connectivity paths for 6 genes. Left: Expression tracing for a single cell is shown with the code-string 000100. Right: Connectivity paths (patina lines) of a healthy control, N02 and a prostate cancer (PCa) patient, #40. Cells sharing the same code-string are highlighted with light-blue background. Nineteen code-strings are present in N02 (total 32 cells) while 13 code-strings are found in #40 (total 40 cells). Additional connectivity maps are shown in FIG. 5.

FIGS. 7A-7B. Clonal analysis of binary code-strings in patient subgroups. (a), Frequency of code-strings in control and patient groups. Common code-strings for each class are underlined, green-normal control, light blue—benign prostate hyperplasia (BPH), pink—high-grade prostatic intraepithelial neoplasia (HGPIN), red—prostate cancer (PCa-I, II, and III), and grey—remaining code-strings in Panel b. (b), Clonal sizes of urinary prostate cells in each patient are marked by colors based on different code-string classes.

FIG. 8. Gene expression analysis of urinary prostate cells in patient subgroups. Microfluidic PCR analysis was conducted in 1220 cells from normal controls, patients with benign prostate hyperplasia (BPH), high-grade prostatic intraepithelial neoplasia (HGPIN), and prostate cancer subgroups (PCa-I, -II, and -III). Normalized expression values range from 0 to 35. Representative examples of gene expression are shown here, and additional single-cell expression data are presented in FIG. 6. Violin plots (bottom) display expression distribution patterns and median values of cells in control and patient subgroups. *P<0.05, **P<0.01, ***P<0.001.

FIG. 9. Illustration of methods for establishing biomarkers for use in single cell biomarker profiling.

### DESCRIPTION

The use of single cell analysis of prostate cancer patient urine samples improves the sensitivity and specificity for prostate specific antigen (PSA) and DRE screening for early prostate cancer diagnosis. The current diagnosis of prostate cancer relies primarily on increased blood prostate specific antigen (PSA) and abnormal digital rectal examination (DRE). These two methods have limits on sensitivity and specificity for the detection of prostate cancer. Evaluation of PSA level in the serum is an indirect and secondary measurement of elevated PSA in the prostate cancer. An inherent limitation of DRE is that only 85 percent of cancers arise peripherally where they can be detected with a finger examination. Within a threshold value of 4 ng/ml, around 15% of men will have prostate cancer that goes undetected, most of whom will have potentially curable disease. The false positives and negatives create unnecessary personal anxiety, increase medical expense, and leave cancerous patients untreated.

Certain aspects include one or more steps selected from (a) fixing of urine samples upon their collection, and (b) single-cell analysis of PSA and PSMA expressions on cells in urine using single cell isolation techniques, such as a DEPArray™ (Silicon Biosystems) as a screen tool for detection of prostate cancer. DEPArray™ technology is based on moving dielectrophoresis cages, to individually sort cells out of a suspension of a relatively small number of cells. The system's core is a chip where an array of individually controllable cages of A/C electrical field is formed. Each cell in suspension is trapped into a cage and numbered. Selected cells then can be individually moved and collected through a software calculated pathway.

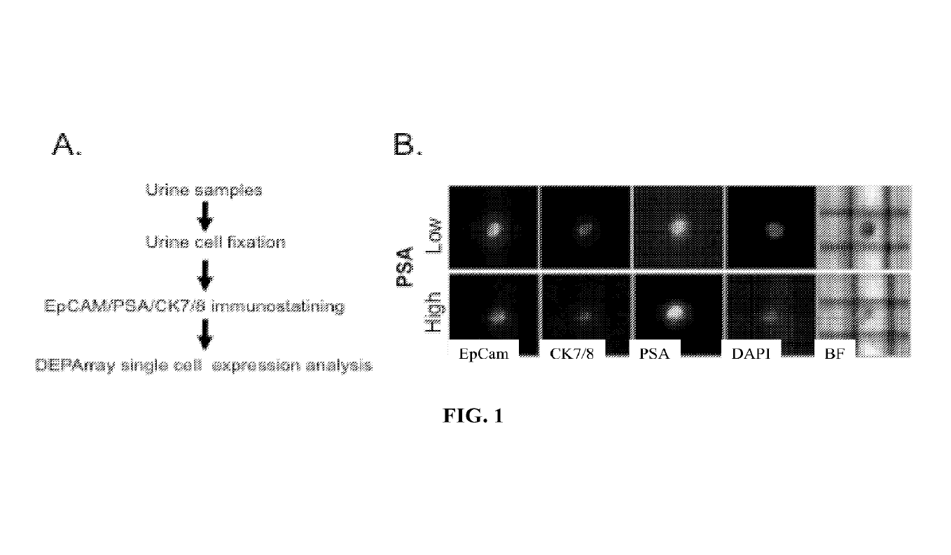

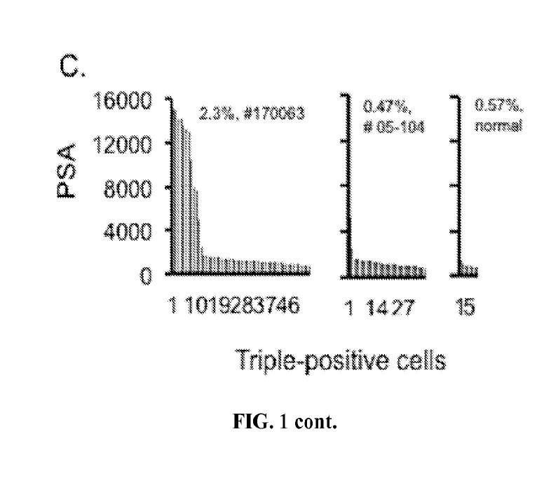

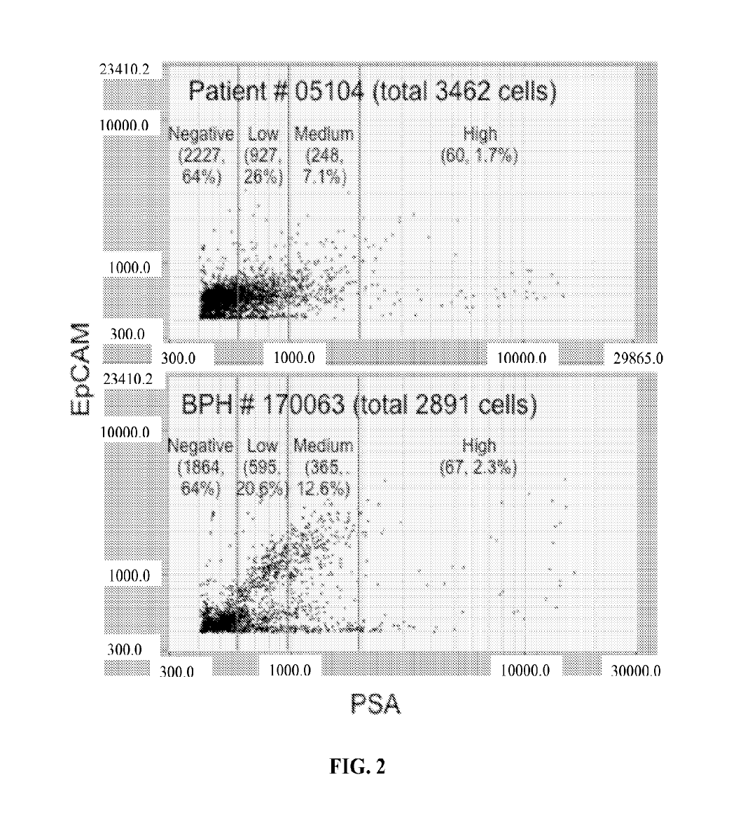

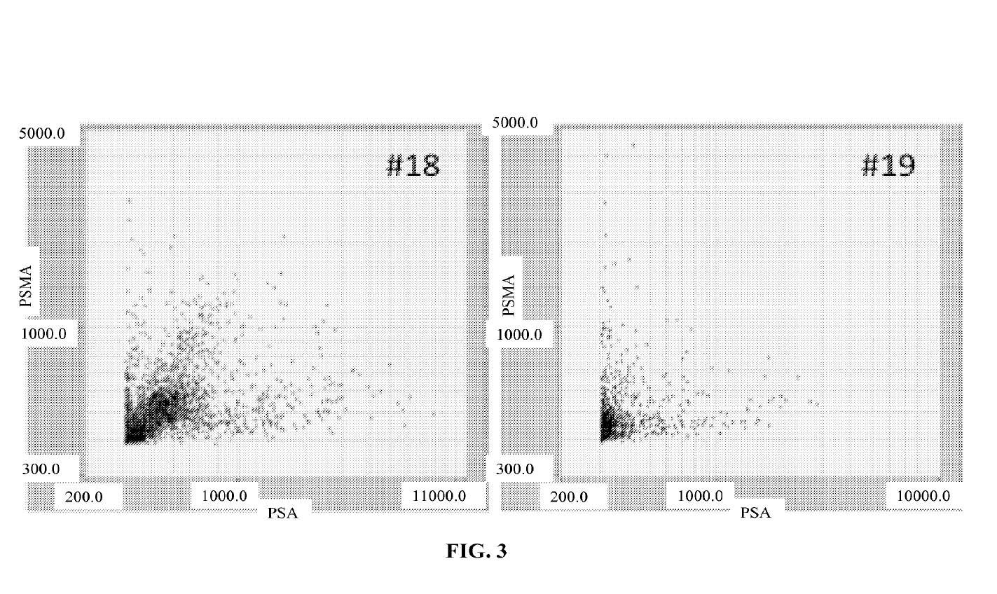

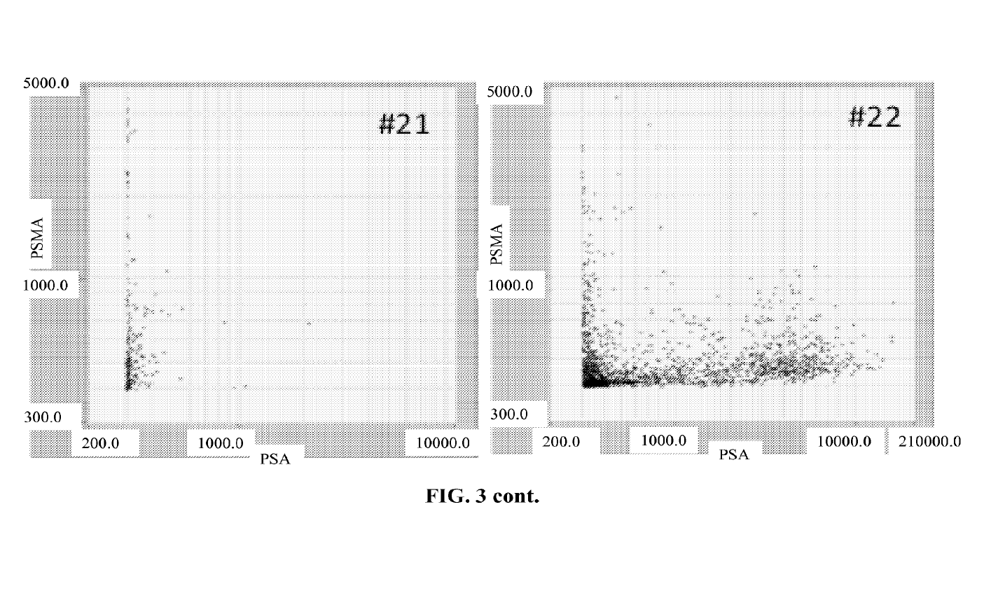

Studies demonstrate that single cells from urine samples with heterogeneous PSA expressions can serve as biomarkers for diagnosis of prostate cancer (FIG. 1 and FIG. 2).

### I. SINGLE CELL ANALYSIS AND DIGITAL BIOPSY™

Isolation of single cells. Biological samples can be collected in a needle, container, syringe, cup, bag, or other suitable collection device. In certain aspects the biological sample is contacted with a preservative. Typically biological samples are cooled (kept on ice or refrigerated) and/or processed immediately. In certain aspects cellular components are precipitated by centrifugation. In certain aspects a tissue sample is dispersed and optionally clarified or filtered prior to centrifugation. After centrifugation the supernatant can be removed leaving a cell pellet in the container. Cell pellets are suspended in a buffer solution and transferred to a second centrifuge tube (e.g., a low-retention centrifuge tube) and spun again to pellet the cells. The wash and centrifugation steps can be repeated multiple times. Cell pellets are suspended in a trypsin containing buffer to dissociate cell aggregates followed by neutralization with an appropriate solution. The neutralized solution is then centrifuged. After the supernatant is removed, cell pellets are suspended in labeling buffer and labeled with one or more primary antibodies that specifically bind to a target cell. The labeled cells are collected and washed to remove unbound primary antibodies. In certain aspects a secondary antibody is provided in an appropriate solution at an appropriate dilution. The cells are collected, e.g., centrifuged and washed with an appropriate buffer to remove the secondary antibody. The cells are suspended in a buffer compatible with immunostaining and examined for immunostaining Single cells identified by a particular antibody binding profile are isolated. In certain aspects the cells can be isolated using a combined micromanipulator-microinjector system (CM2S) (Chen et al. Prostate 73, 813-826 (2013)). The isolated cell is lysed in reaction buffer and either analyzed or stored for later analysis, e.g., frozen.

Single-cell microfluidic PCR. In certain aspects microfluidics based RT-PCR can be used to amplify target nucleic acids. Single-cell microfluidics-based RT-PCR analysis is carried out using appropriate components. A portion a single cell lysate is subjected to PCR amplification using appropriate primers for one or more genes and a control gene. In certain aspects genomic contamination is reduced by incubation of the lysate with DNase I solution. PCR primers of selected genes for expression profiling can be selected from known primer sequences or designed using available computer software. A primer mixture for each panel is prepared in buffer by pooling all the primers of each panel.

Reverse transcription (RT) and pre-amplification are performed on a single-cell total RNA reaction mix comprising a reverse transcriptase and thermocycle DNA polymerase and a primer mix. RT is performed for a selected time period and then inactivated. Pre-amplification follows the RT reaction. Excessive primers in pre-amplification are removed by digestion with an exonuclease. Pre-amplified products can be diluted prior to PCR. The pre-amplified products are subjected to PCR.

A. Single-Cell Expression Data Analysis.

Data normalization. Expression levels of 35 genes, obtained as threshold cycle (Cₜ) values, were normalized to that of the control reference gene UBB and displayed as −ΔΔCₜ values²⁵. The UBB gene was used as a control because its mRNA was found to be highly stable in single prostate cells in our previous microfluidics-based PCR assays¹⁶. We only selected cells that expressed UBB at a threshold of Cₜ≦30 after pre-amplification, assuming that these cells expressing robust expression of UBB are less likely to contain degraded RNA. The −ΔΔCₜ values ranged from the lowest expression level of 0 to the highest expression level of 35, which were used to construct expression heatmaps (see FIGS. 1 and 2 and Supplementary FIG. 2).

Violin plot analysis. A violin expression plot, which combines a box plot and a rotated kernel density plot¹⁷, were constructed for each gene to determine clonal distributions of gene expression in a given population of prostate cells. The density trace is plotted symmetrically to the left and the right the vertical box plot, and there is no difference in these density traces other than the direction in which they extend. Median expression levels of these genes from urinary single cells isolated in 1) normal controls and patients diagnosed with 2) benign prostate hyperplasia (BPH), 3) prostatic intraepithelial neoplasia (PIN) and 4) prostate cancer were analyzed using one-way ANOVA and unpaired Student's t test using R. A P value of <0.05 is considered as statistically significant.

Parallel coordinate plot analysis. Expression patterns of 6 genes in urinary single cells were visualized in parallel coordinate plots using the software of GGobi data visualization system²⁶. Each parallel coordinate plot was composed of points and lines. The points, referring to cells (total 1,220 cells), were arranged from the left to the right for each gene according to its gene expression values from the least to the highest. The lines linked to these points displayed expression connectivity among these 6 genes. Expression connectivities of selected cells for each patient were highlighted in patina color, and all the rest were in brown color (see explanations in the main text).

In silico analysis of gene expression. Gene expression (RNA-seq) data of adjacent normal (n=37) and primary PCa (n=140) used for this study were obtained from The Cancer Genome Atlas (TCGA). In order to display the expression level of selected genes in the same heat map, TCGA data were adjusted using Normalize Genes/Rows function in the software of MultipleExperiment Viewer 4.8. This process standardized gene expression values using the mean and the standard deviation of the row of the matrix to which the gene belongs. The difference between Prostate samples and Normal samples was further compared by Student's t-test using Prism 6 (GraphPad Software, La Jolla, Calif.). A P value of <0.05 is considered as statistically significant.

B. Biopsy Graphical Display

Certain embodiments include the graphical display of analysis of a population of single cells. In certain aspect this graphical display is called DIGITAL BIOPSY™. The graphical methods are used to convey the results of the analysis in a simple easy to read format that has the general appearance of histology section. Steps for preparing such a graphical display include one or more of: (a) Analyzing a population of single cells to select target cells for biomarker profiling. (b) analyzing the selected cells to determine the expression level of components of a biomarker panel (e.g., protein or nucleic acid biomarkers). (c) Quantization of biomarker data to a binary code where “0” or “1” represents gene underexpression or overexpression, respectively. A violin plot can be used to identify appropriate cutoff point for assignment of binary value. (d) Using a parallel coordinate plot (PCP) for visualizing the range of results from a batch of clinical specimens. A particular order of biomarkers are used to represent the binary results for a biomarker panel, which are displayed as a binary clone for a particular cell with a particular binary code, e.g., with a six marker panel a six number binary code is established (e.g., all cells having a binary code 001100 are designated as the same binary clone). For a six marker biomarker panel there are 2⁶=64 potential binary codes/clones. (e) Each unique binary code/clone is quantified and the frequency of detection of each binary code/clone is represented by a colored circle positioned within a boundary. The circle size is proportional to the number of cells detected for a particular binary code/clone. (f) The analysis and graphical depiction of the results correlates to the clinical diagnosis of each individual tested and results in a powerful and easy to interpret display of pathologic significance. The analysis and/or graphical method can be used to test a patient's disease status as well as to monitor a patient over time, as the disease may progress. As an example of the success of the described method, the inventors have identified the specific binary gene expression clones that correlate with more advanced (Stage II and Stage III) prostate cancer vs normal controls, BPH and Stage I.

The graphical display of biomarker panel results can be used for analysis and display of various biomarker panels for diseases including cancer. In certain aspects the method can be used on various clinical specimens such as tissue, blood, urine, serum, saliva, and sweat samples. The only requirement of the sample is that it contains target cells and can be dispersed to include a population of single cell targets.

### II. ANALYSIS AND GRAPHICAL DISPLAY IN PROSTATE CANCER

Prostate cancer is a form of cancer that develops in the prostate, a gland in the male reproductive system. The cancer cells may metastasize (spread) from the prostate to other parts of the body, particularly the bones and lymph nodes. Prostate cancer can cause pain, difficulty in urinating, problems during sexual intercourse, or erectile dysfunction. Other symptoms can potentially develop during later stages of the disease.

Rates of detection of prostate cancers vary widely across the world, with South and East Asia detecting less frequently than in Europe, and especially the United States. Prostate cancer tends to develop in men over the age of fifty. Many factors, including genetics and diet, have been implicated in the development of prostate cancer. The presence of prostate cancer may be indicated by symptoms, physical examination, prostate specific antigen (PSA), or biopsy. There is controversy about the accuracy of the PSA test and the value of screening. Suspected prostate cancer is typically confirmed by taking a biopsy of the prostate and examining it under a microscope. Further tests, such as CT scans and bone scans, may be performed to determine whether prostate cancer has spread.

Treatment options for prostate cancer with intent to cure are primarily surgery, radiation therapy, and proton therapy. Other treatments, such as hormonal therapy, chemotherapy, cryosurgery, and high intensity focused ultrasound (HIFU) also exist, depending on the clinical scenario and desired outcome.

The age and underlying health of the man, the extent of metastasis, appearance under the microscope, and response of the cancer to initial treatment are important in determining the outcome of the disease. The decision whether or not to treat localized prostate cancer (a tumor that is contained within the prostate) with curative intent is a patient trade-off between the expected beneficial and harmful effects in terms of patient survival and quality of life.

### III. METHODS OF DETECTING PROSTATE CANCER

The single-cell approach described herein reduces the possibility of false positives and false negatives. To that end, the methods would assist in early detection of prostate cancer, improve human health, and decrease unnecessary medical expenses. The invention utilizes much less invasive method with the urine samples that are usually collected post-DRE. The methods can be used in combination with other prostate cancer screening methods such as PSA levels in the blood.

The methods described herein are less invasive, e.g., urine samples are collected post-DRE. In combination with PSA in the blood, single cell analysis using post-DRE urine samples can be used for detecting prostate cancer. A sufficient number of prostate cells are found in urine after DRE for conducting single cell analysis.

The method includes one or more of the following steps. Urine samples and/or other biological samples are collected from a subject. In certain aspects the urine sample is collected after DRE. In certain aspects the urine samples are contacted with a preservative. Cells present in the sample are separated from biological fluids. For example, the cells in the sample are pelleted by centrifugation.

The isolated cells are processed. In certain aspects the cells are contacted with a detectable antibody. The antibody or antibodies include antibodies that bind proteins that are used as a control, a reference, or a biomarker. In certain aspects the antibody is detectably labeled. Detectable labeled refers to the attachment of a moiety to the antibody that can be directly or indirectly detected and/or measured.

The labeled cells can then be isolated and/or sorted. In certain aspects the cells are loaded onto a DEPArray™ for single cell isolation and then BioMark™ molecular profiling device using TBIIR and miRNA gene primer panel.

In certain aspects all or a portion of the cells collected from the sample are fixed. For fixed cells, pellets are washed, fixed, and antibody labeled. Cells are fixed using formaldehyde. The fixed cells are labeled with a detectable antibody. The labeled cells are then sorted and/or isolated and analyzed at the single cell level.

In certain aspects the labeled cells are analyzed using a DEPArray™ in conjunction with DEPArray™ data analysis. Several dozens to thousands of cells isolated from urine are loaded unto DEPArray chips (cat# Silicon Biosystems, Inc) according to manufacturer's protocol. For live cells, the cells were suspended in DMEM+5% FBS+P/S (1×) and in SB115 buffer.

### IV. BIOMARKERS

A biomarker is a biomolecule that is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. As such, they are useful as markers for disease (diagnostics), therapeutic effectiveness of a drug (theranostics) and of drug toxicity.

A biomarker panel can include 2, 3, 4, 5, 6, 7, 8, 9, 10, or more biomarkers. In certain aspects the biomarkers are correlated to particular state, such as normal, benign, or varying degrees of a pathological state.

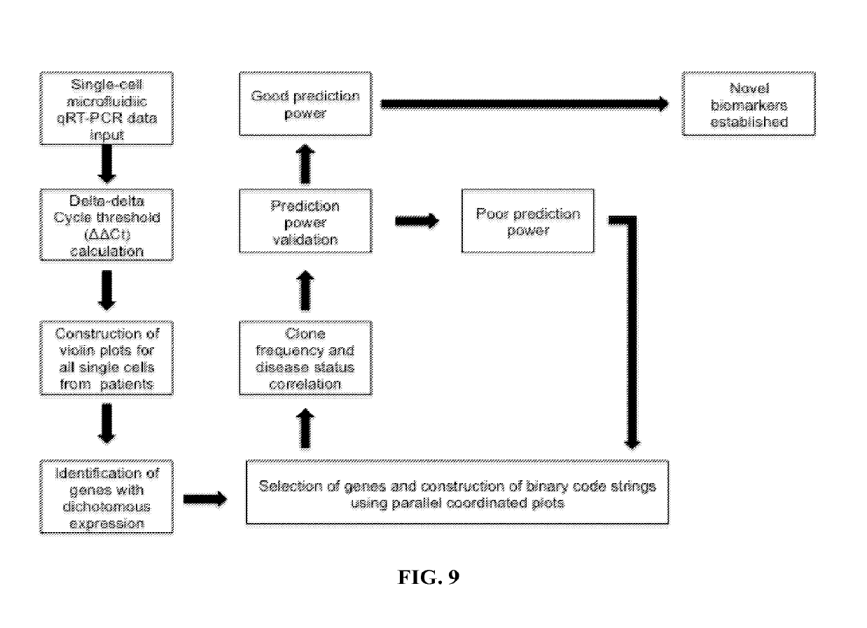

FIG. 9 illustrates an example of a method for establishing biomarkers for use in a single cell biomarker panel. In certain aspects of the method can be implemented using a computer system. A computer system can comprise instructions to receive, analyze, and determine if one or more biomarker or a set of biomarkers are effective in single cell biomarker profile assays. The computer system receives data from single cell PCR assay(s). The computer system calculates the delta-delta cycle threshold (ΔΔCt) for a candidate biomarker. The results of the ΔΔCt are transformed by the system into violin plots that include all single cell results from a given patient. The system identifies which biomarkers are dichotomously expressed. The system selects which biomarkers are dichotomously expressed and uses the selected biomarkers to construct binary code strings using parallel coordinated plots. The system assigns a binary code string associated with a biomarker panel to generate single cell biomarker profile that identifies a clone. The system assesses the correlation between clone frequency and disease status. The system analyzed the strength of the correlation using prediction power validation. If the clone frequency is a poor predictor then the system selects a new set of genes and constructs new binary code strings and then analyzes the new clones for correlation. If the clone is a good predictor then the system selects this code string as an established single cell biomarker panel.

Prostate Specific Antigen (PSA). PSA is a peptidase of the kallikrein family and a differentiation antigen of the prostate. Alternate names include gamma-seminoprotein, kallikrein 3, seminogelase, seminin, and P-antigen.

Prostate Specific Membrane Antigen (PSMA). PSMA, also known as Glutamate carboxypeptidase II, is a type 2 integral membrane glycoprotein found in prostate and a few other tissues. PSMA is expressed on tumor cells as a noncovalent homodimer.

Epithelial cell adhesion molecule (EpCAM). EpCAM, also known as TACSTD1 (tumor-associated calcium signal transducer 1) and CD326 (cluster of differentiation 326), is a pan-epithelial differentiation antigen that is expressed on almost all carcinomas. It has been used as an immunotherapeutic target in the treatment of gastrointestinal, urological and other carcinomas. EpCAM is a carcinoma-associated antigen and is a member of a family that includes at least two type I membrane proteins. This antigen is expressed on most normal epithelial cells and gastrointestinal carcinomas and functions as a homotypic calcium-independent cell adhesion molecule.

Cytokeratins (CK7/8). Cytokeratins constitute homology groups I and II. The nomenclature chosen in 1982 by Moll and Franke assign ranges from 1 to 8 for type I cytokeratins (neutral or alkaline) and from 9 to 12 for type II cytokeratins (acids). Cytokeratin 7 is a basic cytokeratin which is localized in most of glandular and transitional epithelial, but not in stratified squamous epitheliums. Cytokeratin 8 belongs to type B subfamily (alkaline) high molecular weight cytokeratins.

### V. CANCER TREATMENTS

In certain aspects, there may be provided methods for treating a subject determined to have cancer and with a predetermined expression profile of one or more biomarkers disclosed herein. In a further aspect, biomarkers and related systems, including biomarker expression profiles correlating to a particular DIGITAL BIOPSY™ binary code/clone as described herein, that can establish a prognosis of cancer patients can be used to identify patients who may benefit from conventional single or combined modality therapy. In the same way, the invention can identify those patients who do not benefit from such conventional single or combined modality therapy and can offer them alternative treatment(s).

In certain aspects of the present invention, conventional cancer therapy may be applied to a subject wherein the subject is identified or reported as having a good prognosis based on the assessment of the biomarkers as disclosed. On the other hand, at least an alternative cancer therapy may be prescribed, as used alone or in combination with conventional cancer therapy, if a poor prognosis is determined by the disclosed methods, systems, or kits.

Conventional cancer therapies include one or more selected from the group of chemical or radiation based treatments and surgery. Chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

Radiation therapy causes DNA damage and has been used extensively, including what are commonly known as γ-rays, X-rays, and/or the directed delivery of radioisotopes to tumor cells or organs. Other forms of DNA damaging factors are also contemplated such as microwaves and UV-irradiation. Dosage ranges for X-rays range from daily doses of 50 to 200 roentgens for prolonged periods of time (3 to 4 wk), single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and/or a chemotherapeutic or radiotherapeutic agent are delivered to a target cell or are placed in direct juxtaposition with the target cell. In certain aspects both agents are delivered to a cell in a combined amount effective to kill the cell or prevent it from dividing.

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies. Curative surgery includes resection in which all or part, of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery).

Laser therapy is the use of high-intensity light to destroy tumor cells. Laser therapy affects the cells only in the treated area. Laser therapy may be used to destroy cancerous tissue and/or relieve a blockage when the cancer cannot be removed by surgery. The relief of a blockage can help to reduce symptoms.

Photodynamic therapy (PDT), a type of laser therapy, involves the use of drugs that are absorbed by cancer cells; when exposed to a special light the drugs become active and destroy the cancer cells.

Upon excision of part of all of cancerous cells, tissue, or tumor, a cavity may be formed in the body. Treatment may be accomplished by perfusion, direct injection or local application of the area with an additional anti-cancer therapy.

Alternative cancer therapy includes immunotherapy, gene therapy, hormonal therapy or a combination thereof. Subjects identified with poor prognosis using the present methods may not have favorable response to conventional treatment(s) alone and may be prescribed or administered one or more alternative cancer therapy per se or in combination with one or more conventional treatments.

### VI. COMPUTER IMPLEMENTATION

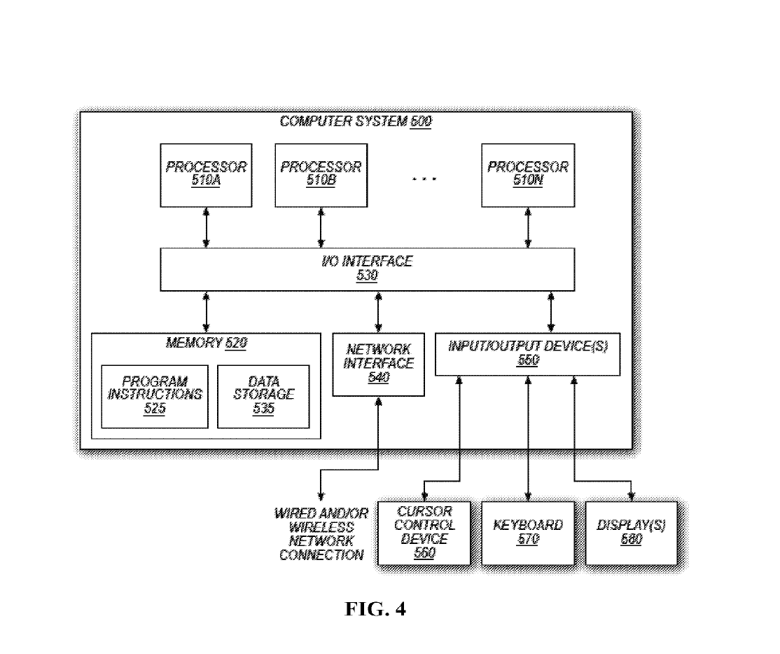

Embodiments of assays or methods described herein or the analysis thereof may be implemented or executed by one or more computer systems. One such computer system is illustrated in FIG. 4. In various embodiments, computer system may be a server, a mainframe computer system, a workstation, a network computer, a desktop computer, a laptop, or the like. For example, in some cases, the analysis described herein or the like may be implemented as a computer system. Moreover, one or more of servers or devices may include one or more computers or computing devices generally in the form of a computer system. In different embodiments these various computer systems may be configured to communicate with each other in any suitable way, such as, for example, via a network.

As illustrated, the computer system includes one or more processors 510 coupled to a system memory 520 via an input/output (I/O) interface 530. Computer system 500 further includes a network interface 540 coupled to I/O interface 530, and one or more input/output devices 550, such as cursor control device 560, keyboard 570, and display(s) 580. In some embodiments, a given entity (e.g., analysis of subjects for trypanosome infection and/or cardiomyopathy) may be implemented using a single instance of computer system 500, while in other embodiments multiple such systems, or multiple nodes making up computer system 500, may be configured to host different portions or instances of embodiments. For example, in an embodiment some elements may be implemented via one or more nodes of computer system 500 that are distinct from those nodes implementing other elements (e.g., a first computer system may implement an assessment of a hybrid latent variable assessment or system while another computer system may implement data gathering, scaling, classification etc.).

In various embodiments, computer system 500 may be a single-processor system including one processor 510, or a multi-processor system including two or more processors 510 (e.g., two, four, eight, or another suitable number). Processors 510 may be any processor capable of executing program instructions. For example, in various embodiments, processors 510 may be general-purpose or embedded processors implementing any of a variety of instruction set architectures (ISAs), such as the x86, POWERPC®, ARM®, SPARC®, or MIPS® ISAs, or any other suitable ISA. In multi-processor systems, each of processors 510 may commonly, but not necessarily, implement the same ISA. Also, in some embodiments, at least one processor 510 may be a graphics-processing unit (GPU) or other dedicated graphics-rendering device.

System memory 520 may be configured to store program instructions and/or data accessible by processor 510. In various embodiments, system memory 520 may be implemented using any suitable memory technology, such as static random access memory (SRAM), synchronous dynamic RAM (SDRAM), nonvolatile/Flash-type memory, or any other type of memory. As illustrated, program instructions and data implementing certain operations, such as, for example, those described herein, may be stored within system memory 520 as program instructions 525 and data storage 535, respectively. In other embodiments, program instructions and/or data may be received, sent or stored upon different types of computer-accessible media or on similar media separate from system memory 520 or computer system 500. Generally speaking, a computer-accessible medium may include any tangible storage media or memory media such as magnetic or optical media—e.g., disk or CD/DVD-ROM coupled to computer system 500 via I/O interface 530. Program instructions and data stored on a tangible computer-accessible medium in non-transitory form may further be transmitted by transmission media or signals such as electrical, electromagnetic, or digital signals, which may be conveyed via a communication medium such as a network and/or a wireless link, such as may be implemented via network interface 540.

In an embodiment, I/O interface 530 may be configured to coordinate I/O traffic between processor 510, system memory 520, and any peripheral devices in the device, including network interface 540 or other peripheral interfaces, such as input/output devices 550. In some embodiments, I/O interface 530 may perform any necessary protocol, timing or other data transformations to convert data signals from one component (e.g., system memory 520) into a format suitable for use by another component (e.g., processor 510). In some embodiments, I/O interface 530 may include support for devices attached through various types of peripheral buses, such as a variant of the Peripheral Component Interconnect (PCI) bus standard or the Universal Serial Bus (USB) standard, for example. In some embodiments, the function of I/O interface 530 may be split into two or more separate components, such as a north bridge and a south bridge, for example. In addition, in some embodiments some or all of the functionality of I/O interface 530, such as an interface to system memory 520, may be incorporated directly into processor 510.

Network interface 540 may be configured to allow data to be exchanged between computer system 500 and other devices attached to a network, such as electronic medical records systems, laboratory data reporting systems, health information exchange networks or other computer systems, or between nodes of computer system 500. In various embodiments, network interface 540 may support communication via wired or wireless general data networks, such as any suitable type of Ethernet network, for example; via telecommunications/telephony networks such as analog voice networks or digital fiber communications networks; via storage area networks such as Fiber Channel SANs, or via any other suitable type of network and/or protocol.

Input/output devices 550 may, in some embodiments, include one or more display terminals, keyboards, keypads, touch screens, scanning devices, voice or optical recognition devices, or any other devices suitable for entering or retrieving data by one or more computer system 500. Multiple input/output devices 550 may be present in computer system 500 or may be distributed on various nodes of computer system 500. In some embodiments, similar input/output devices may be separate from computer system 500 and may interact with one or more nodes of computer system 500 through a wired or wireless connection, such as over network interface 540.

As shown in FIG. 4, memory 520 may include program instructions 525, configured to implement certain embodiments described herein, and data storage 535, comprising various data accessible by program instructions 525. In an embodiment, program instructions 525 may include software elements of embodiments illustrated herein. For example, program instructions 525 may be implemented in various embodiments using any desired programming language, scripting language, or combination of programming languages and/or scripting languages (e.g., C, C++, C#, JAVA®, JAVASCRIPT®, PERL®, etc). Data storage 535 may include data that may be used in these embodiments. In other embodiments, other or different software elements and data may be included.

A person of ordinary skill in the art will appreciate that computer system 500 is merely illustrative and is not intended to limit the scope of the disclosure described herein. In particular, the computer system and devices may include any combination of hardware or software that can perform the indicated operations. In addition, the operations performed by the illustrated components may, in some embodiments, be performed by fewer components or distributed across additional components. Similarly, in other embodiments, the operations of some of the illustrated components may not be performed and/or other additional operations may be available. Accordingly, systems and methods described herein may be implemented or executed with other computer system configurations.

### VII. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Single Cell Analysis of Post-DRE Urine Samples

Materials and Methods

About 20 ml or more post-digital rectal examination urine samples were centrifuged in 50 ml conical tubes at 400×g at 4° C. for 5 min. The urine supernatant is expirated until ˜2 ml of supernatant is left lest the urine cells in the pellets may be sucked out. The remaining supernatant is continuously removed using P1000 pipetteman. The pellets were subjected to two different processing. For live cell processing, then the pellets were subject washings and labeling with antibodies, for example:

1. Cell pellets are suspended in 1 ml 1× PBS and transfer onto a 1.5 ml centrifuge tube and spun in a bench microcentrifuge 400×g at 4° C. for 5 min.

2. Repeat step 1.

3. Add 100 μl 0.05% trypsin at 37° C. for 10 min and spin in a bench microcentrifuge 400×g at 4° C. for 5 min.

4. Suspend cell pellets in DMEM+5% FBS+P/S (1×) and label with and label polyclonal rabbit α-PSA (1:100, Dako, #A0562), α-hPSMA/FOLH1-APC (1:10, R&D system, #FAB4234A). Incubate on ice for 15 min with light proof (aluminum foil).

5. Microcentrifuge 400×g at 4° C. for 5 min and wash with 1 ml DMEM+5% FBS+P/S (1×) twice to remove the primary (1°) antibodies.

6. Apply secondary (2°) Antibodies (anti-rabbit IgG-Cy3, 500× dilution) in 200 μl DMEM+5% FBS+P/S (2%)+0.5 μg/ml DAPI at RT for 15 min.

7. Microcentrifuge 400×g at 4° C. for 5 min and wash with 1 ml DMEM+5% FBS+P/S (1×) twice to remove the 2° antibodies.

8. Suspend in 20 μl DMEM+5% FBS+P/S (1×)

9. Check the immunostaining of cells under an Evos fl inverted microscope.

10. The cells are ready to load onto DEPArray for single cell isolation and then BioMark molecular profiling using TBIIR and miRNA gene primer panel.

For fixed cells processing, the pellets were subject to washings, fixation, and antibody labeling as below:

1. Centrifuge urine sample (˜20 ml) in a 50 ml conical tube at 400×g at 4° C. for 5 min.

2. Remove the urine supernatant gently without disturbing cell pellets.

3. Suspend cell pellets with 1 ml 1× PBS and transfer onto a 1.5 ml centrifuge tube and spin at 400×g at 4° C. for 5 min.

4. Remove the supernatant and add 100 μl 0.05% trypsin and incubated at 37° C. for 10 min.

5. Spin the tube at 400×g at 4° C. for 5 min and removed the supernatant.

6. Resuspend the cells in 200 μl 1×PBS

7. Fix cells in urine with 2% formaldehyde for 20 min at room temperature.

8. Spin the tube in a bench microcentrifuge for 20 seconds.

9. Suspend cell pellets with 1 ml PBS+5% FBS+0.2% tween 20 and spin in a bench microcentrifuge for 20 seconds.

10. Repeat step 9.

11. Suspend cell pellets in 100 μl PBS+5% FBS+0.2% tween 20 and label cells with polyclonal rabbit α-PSA (1:100, DAKO, #A0562), α-hPSMA/FOLH1-APC (1:10, R&D system, #FAB4234A) and incubate on ice for 15 min with light proof (aluminum foil).

12. Spin in a bench microcentrifuge for 20 seconds.

13. Wash with 1 ml PBS+5% FBS+0.2% tween 20 to remove the 1° antibodies.

14. Apply 2° Antibodies (anti-rabbit IgG-Cy3) with 500× dilution in 100 μl PBS+5% FBS+0.2% tween 20+0.5 μg/ml DAPI (1:100 dilution) at RT for 15 min.

15. Spin in a bench microcentrifuge for 20 seconds and wash with 500 μl SB115 buffer twice to remove the 2° antibodies.

16. Suspend in 20 μl SB115 buffer

17. Check the immunostaining of cells under an Evos fl inverted microscope.

18. The cells are ready to load onto DEPArray and subject to single cell analysis according to the protocol from Silicon Biosystems, Inc.

DEPArray Data Analysis:

About several dozens to three thousands urine cells were loaded unto DEPArray chips (cat# Silicon Biosystems, Inc) according to manufacturer's protocol. For live cells, the cells were suspended in DMEM+5% FBS+P/S (1×) or in SB115 buffer.

### Example 2

### Analysis and Graphical Display in Prostate Cancer

Single-cell analyses have revealed diverse patterns of gene expression in a cancer cell population (Meacham and Morrison, Nature 501, 328-337 (2013); Almendro et al., Annu Rev Pathol 8, 277-302 (2013)). The inventors describe a class of genes whose expression patterns can be reduced to binary codes at the single-cell level. Of 34 prostate cancer (PCa)-related genes examined in urinary cells originating from the prostate gland, six loci display the dichotomous characteristic that is coded as 0 for low and 1 for high expression, respectively. When arranging these genes in an order CXCL6-TGFBR2-GSK3B-CDKN1C-GATA3-EIF4EBP1, the inventors identify 64 (2*2*2*2*2*2) binary codes in 1220 single cells analyzed. Parallel coordinate plot (Swayne et al., Comput Stat Data An 43, 423-44 (2003)) is used to connect binary codes into a string (e.g., 111111, 101010, 010101, or 000000) for a single cell. Whereas these combinatorial codes are diverse in normal controls, unique code-strings are found in PCa patients. Furthermore, these code-strings represent different clonal populations of patient subgroups. High expression levels of tumor-promoting genes, including EPCAM and E2F1, are found in one subgroup, suggesting active clonal expansions of their cancer cells. Thus, the digital rendering of complex expression patterns enables identification of PCa cells in urine, providing a diagnostic adjunct to biopsy for cancer detection and risk assessment. This approach can also be used for clonal analysis of exfoliated cells for other diseases.

Epithelial cells exfoliated from the prostate gland are sometimes released into the urethra, thus appearing in urine (Ploussard and de la Taille, Nat Rev Urol 7, 101-09 (2010); Crawford et al., Diagnostic Performance of PCA3 to Detect Prostate Cancer in Men with Increased Prostate Specific Antigen: A Prospective Study of 1,962 Cases. J Urol (2012)). During the neoplastic process, a great number of abnormal prostate cells are exfoliated, providing a unique opportunity for cancer detection (Truong et al., J Urol 189, 422-29 (2013)). Previous analyses have confirmed cancer cells of the prostate origin in urine (Truong et al., J Urol 189, 422-29 (2013); Fujita et al. Hum Pathol 40, 924-33 (2009)), and prostate cancer antigen 3 (PCA3) is a urinary biomarker for PCa (Crawford et al. Diagnostic Performance of PCA3 to Detect Prostate Cancer in Men with Increased Prostate Specific Antigen: A Prospective Study of 1,962 Cases. J Urol (2012)). However, PCA3 has only moderate sensitivity and specificity for PCa detection (Crawford et al. Diagnostic Performance of PCA3 to Detect Prostate Cancer in Men with Increased Prostate Specific Antigen: A Prospective Study of 1,962 Cases. J Urol (2012); Whitman et al. J Urol 180, 1975-78 (2008)). Furthermore, PCa cells exfoliated in urine likely express diverse levels of PCA3, and the accurate measurement is frequently hampered when PCa cells are analyzed from a mixed urinary cell populations (Buganim et al. Cell 150, 1209-1222 (2012)).

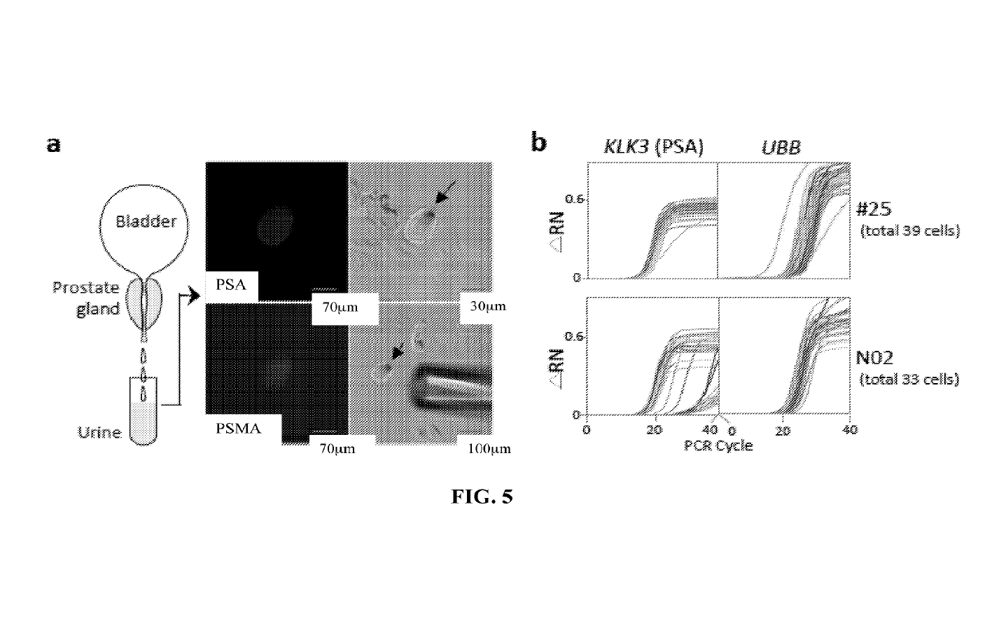

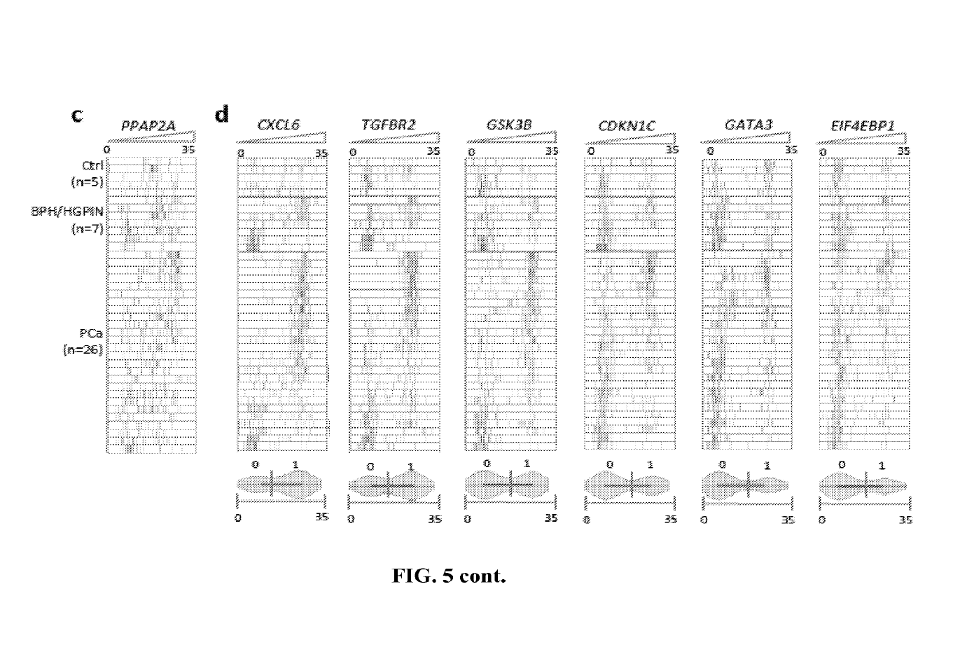

Motivated by the need to improve PCa detection, the inventors developed a method to analyze single-cell expression profiles (FIG. 5a). Exfoliated prostate cells in urine sediment were fluorescently stained with prostate-specific markers, PSA and PSMA (Ben Jemaa et al., J Exp Clin Cancer Res 29, 171 (2010)) and manually retrieved using a micromanipulator device. A total of 1283 exfoliated cells were collected from 33 patients undergoing prostate biopsy and from 5 healthy controls.

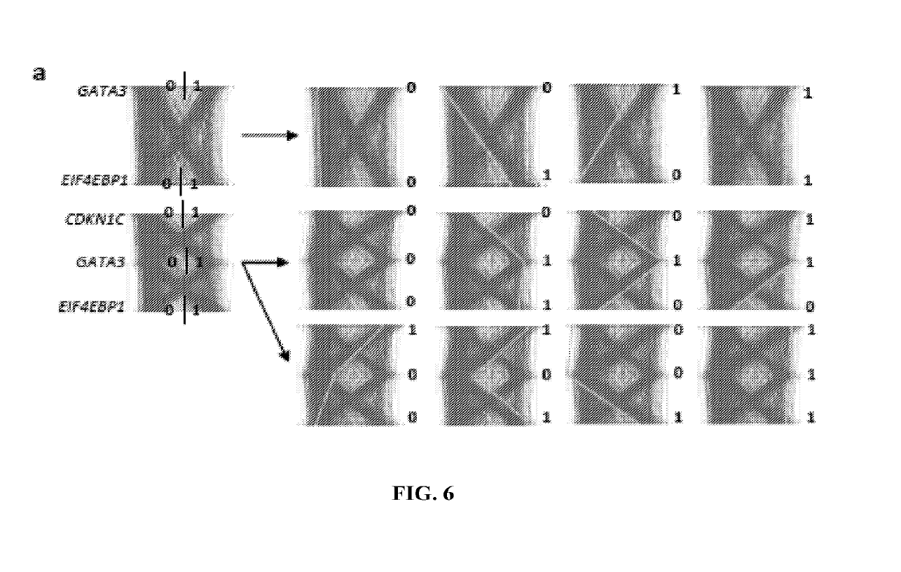

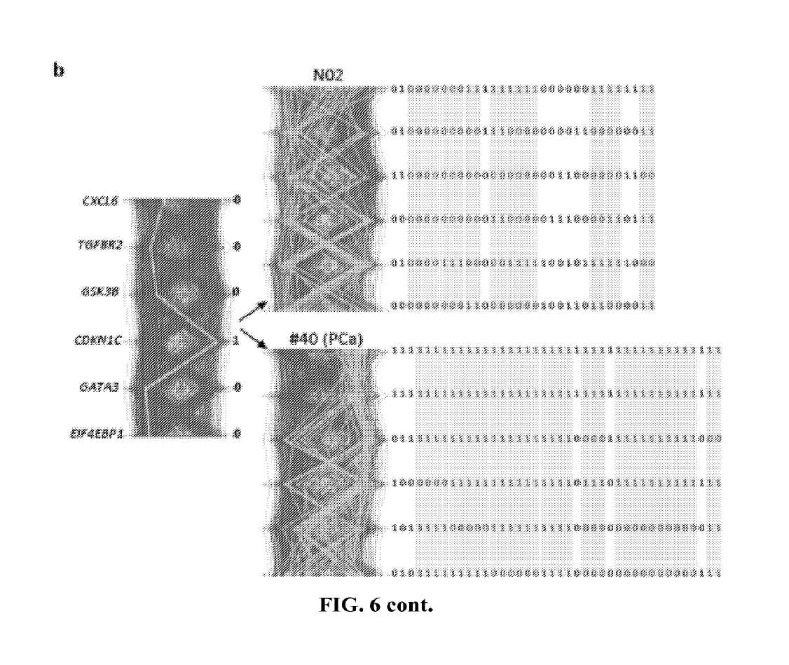

Single cells were subjected to microfluidic PCR analysis of 34 genes known to be aberrantly expressed in PCa (Cai et al. Cancer Cell 20, 457-71 (2011); Begley et al., Cytokine 43, 194-199 (2008)). A total of 1220 urinary prostate cells had robust expression values based on the cycle threshold (Cₜ) of amplification (FIG. 5b). Expression values of genes were normalized to that of a housekeeping gene, Ubiquitin B (UBB), which had stable expression values in prostate and other cell types (Popovici et al., BMC Bioinformatics 10, 42 (2009); Powell et al., PLoS One 7, e33788 (2012); Nikrad et al., Mol Cancer Ther 4, 443-49 (2005); Chen et al. Prostate 73, 813-26 (2013)). Expression levels of 28 genes, such as PPAP2A, varied extensively in single prostate cells (FIG. 5c). However, the remaining six genes exhibited a dichotomous expression pattern at the single-cell level (FIG. 5d). Violin plot analysis (Hintze and Nelson, The American Statistician 52, 181-84 (1998)) confirmed their bimodal expression distributions in prostate cells (FIG. 5d). A binary code system was used to digitize single-cell expression data with 0 as low and 1 as high expression, respectively. Binary codes of these genes were connected with a string for each cell in a parallel coordinate plot (PCP) (Swayne et al., Comput Stat Data An 43, 423-444 (2003)). As shown in FIG. 6a-upper, a map depicts 1220 straight and crisscross strings between two genes, GATA3 and EIF4EBP1, for all cells analyzed. Code-strings-00, 01, 10, and 00 were further shown for four single cells. The third gene, CDKN1C, was added to produce eight possible code-strings 000, 100, 010, 001, 011, 101, 110, and 111 (FIG. 6a-lower). When arranging these genes in this order CXCL6-TGFBR2-GSK3B-CDKN1C-GATA3-EIF4EBP1, all 64 (2*2*2*2*2*2) possible code-strings were identified in 1220 single cells analyzed (FIG. 6b-left). For the normal control N02, 19 code-strings were found in 32 single cells analyzed (FIG. 6b-upper-right). Three code-strings-000000, 000010, and 100010 were repeatedly seen in 14 single cells, suggesting that code-string patterns are not randomly distributed in a population. Of note, the PCP of Patient #40 had a more homogenous pattern than that of N02, with only 13 code-strings being identified in 40 cells (FIG. 6b-lower-right). Six of these code-strings-111011, 111101, 111110, 111111, 110100, and 111100 were frequently seen in the majority (80%) of single cells, suggesting the presence of specific clonal populations in this patient. The inventors constructed 36 PCPs for clonal analysis of these prostate cells.

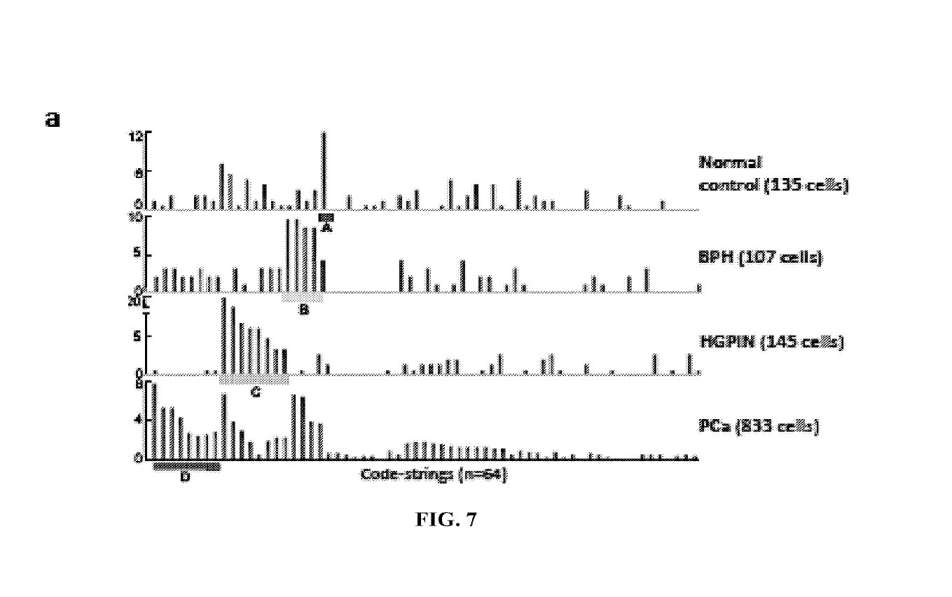

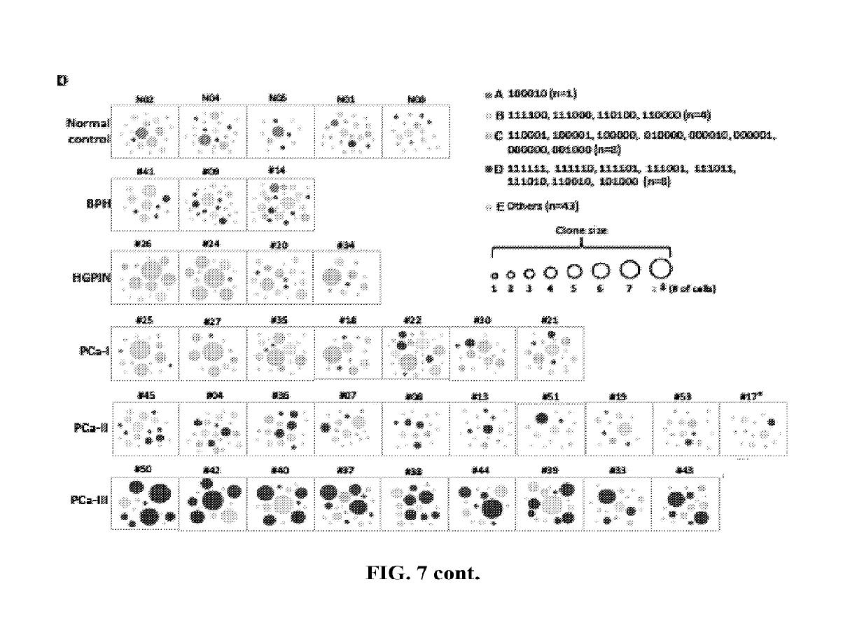

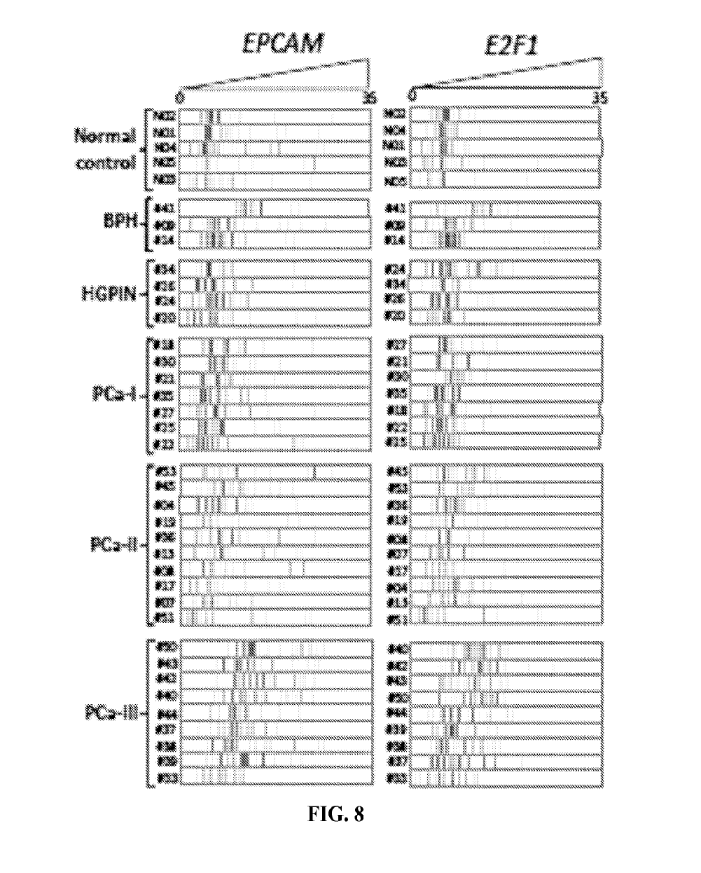

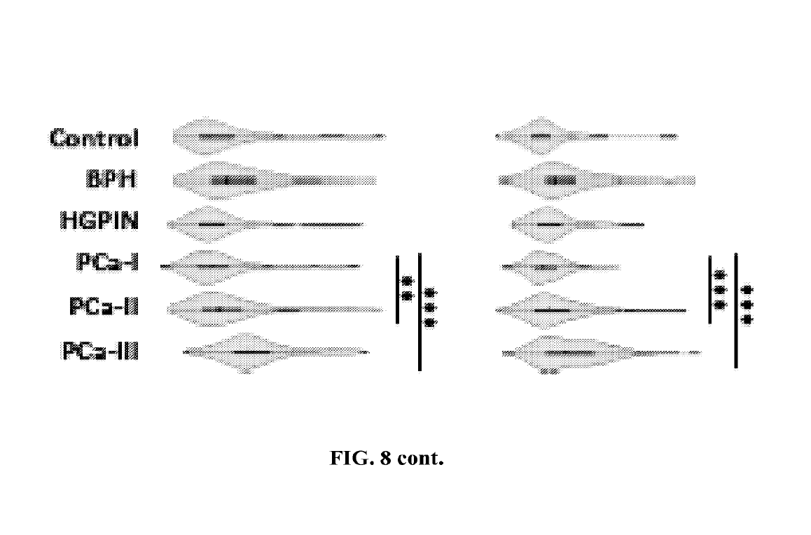

When categorizing code-strings into different classes, 21 code-strings were identified that distinguished different clonal populations of normal control, benign prostate hyperplasia (BPH), high-grade prostatic intraepithelial neoplasia (HGPIN), and PCa-I, -II, and -III subgroups (FIG. 7a). The Class A code-string (n=1) was frequently seen in normal control cells while Class B (n=4) and C (n=8) code-strings were commonly present in BPH and HGPIN groups, respectively (FIG. 7b). Interestingly, Class C code-strings were also found in clonal populations of PCa-I patients, confirming a clonal progression of malignancy from precursor HGPIN in this subgroup (Marusyk and Polyak, Science 339, 528-29 (2013)). Eight other code-strings-111111, 111110, 111101, 111011, 111010, 110010, and 101000 (Class D) were frequently present in PCa-II and -III subgroups. Compared to the former, PCa-III patients had large clonal populations (2-5 clones with ≧3 cells per clone) with Class D code-strings, suggesting active clonal expansions of their cancers. To confirm whether large Class D clones are associated with aggressive disease, single-cell expression data of the aforementioned PCa-related genes were analyzed in these patient subgroups (FIG. 8). Nineteen of 28 genes, including EPCAM and E2F1, were preferentially up-regulated in PCa-III cells compared with two other subgroups, PCa-I and -II (P<0.001). Indeed, EPCAM is known to be highly expressed in high-grade and advanced tumors (Ni et al., Cancer Metastasis Rev 31, 779-91 (2012)) while aberrant expression of E2F1 promotes the development of hormone-independent PCa (Davis et al., Cancer Res 66, 11897-906 (2006)). When examining patients' clinicopathological reports, six (#40, 37, 38, 39, 40, 42, and 44) of nine PCa-III patients had high-grade diseases and/or large tumor volume. However, three PCa-III patients (#33, 43, and 50) appeared to have low-risk PCa based on their biopsy results. As upgrading of low-risk PCa is seen in 30-50% of patients, further follow-up of these patients may confirm them to have aggressive tumors (Chun et al., Eur Urol 49, 820-26 (2006); Pinthus et al., J Urol 176, 979-984; discussion 984 (2006)). One PCa-II patient, #17, who also had aggressive PCa with bone metastasis, carried only a small Class D clone in his urinary prostate cells. Because his urine sample was collected at the time when the patient underwent a hormone ablation therapy, it is speculate that large aggressive clones were eliminated as a result of the therapy. Therefore, this single-cell technique can be offered not only as a diagnostic adjunct to prostate biopsy but also as a non-invasive monitoring of patients' response to treatment in the future.

PSA/PSMA-positive prostate cells were individually retrieved from urine sediment using a micromanipulator device. Cells lysed in reaction buffer were used for one-step CellsDirect™ RT-PCR analysis with the microfluidics system. Normalized values (−ΔΔCt) of genes were obtained for generating expression heat maps, violin graphs, and parallel coordinate plots of single cells. Connectivity paths of genes were converted into binary code-strings for clonal analysis.

Isolation of urinary single cells of the prostate origin. Patient consent for the urine collection was carried out according to IRB protocol approved at the University of Texas Health Science Center San Antonio (UTHSCSA). Urine samples (˜25 mL) collected in a container were transferred onto a 50 ml conical tube and kept on ice for immediate processing. Urinary cellular components were precipitated at 400×g at 4° C. for 5 min. The supernatant was removed gently without disturbing cell pellets. Cell pellets were suspended with 1 mL 1×PBS and transferred onto a 1.5 mL low-retention centrifuge tube and spun down at 400×g for 5 min at 4° C. The wash and centrifugation were repeated. Cell pellets were suspended in 100 mL 0.05% trypsin to dissociate cell aggregates at 37° C. for 10 min and then was neutralized with 500 μl DMEM+5% FBS supplemented with penicillin/streptomycin (P/S), 100 unit/ml and 100 μg/ml, respectively, and centrifuged at 4° C. at 400×g for 5 min. After the supernatant was removed, cell pellets were suspended in 100 μl DMEM+5% FBS+P/S and labeled with polyclonal rabbit α-PSA (v:v=1:100, Dako, #A0562), mouse α-hPSMA(FOLH1)-APC (v:v=1:10, R&D system, #FAB4234A) on ice for 15 min with light proof. The cells were microcentrifuged at 400×g at 4° C. for 5 min and washed with 1 mL DMEM+5% FBS+P/S twice to remove 1° antibodies. A secondary antibody (α-rabbit IgG-Cy3) was applied in a 500-fold dilution with 200 μl DMEM+5% FBS+P/S+0.5 ug/ml DAPI at RT for 15 min on ice. The cells were centrifuged at 400×g for 5 min at 4° C. and subsequently washed with 1 ml DMEM+5% FBS+P/S twice to remove the secondary antibody. The cells were resuspended in 20 μl DMEM+5% FBS+P/S (1×) and examined for immunostaining under an Evos fl inverted microscope. Single PSA/PSMA+ prostate cells were isolated using a combined micromanipulator-microinjector system (CM2S) (Chen et al., Prostate 73, 813-26 (2013)) and lysed in 4 mL 2× reaction buffer (CellDirect™ one step qRT-PCR kit, Invitrogen, Inc) and frozen at −80° C. immediately until further use.

Single-cell microfluidic PCR. Single-cell microfluidics-based RT-PCR analysis was carried out using CellsDirect™ one-step qRT-PCR kit (Invitrogen, Carlsbad, Calif.) with modifications and a microfluidics device, BioMark HD MX/HX system (Fluidigm, South San Francisco, Calif.) (Chen et al., Prostate 73, 813-26 (2013)). Three μl of lysate (˜⅓) of a urinary single cell was subject to PCR amplification using a panel of 34 prostate cancer-related genes and a control gene, Ubiquitin B (UBB). To reduce contamination, genomic DNA from the lysate was degraded in a 18-μl reaction using DNase I (5 units) with 1× DNase I buffer at RT for 5 min. PCR primers of selected genes for expression profiling were selected from the PrimerBank database. A primer mixture (500 nM) for each panel was prepared in TE buffer by pooling all the primers of each panel.

Reverse transcription and pre-amplification were carried out in a 10 μl reaction with 3 μl single-cell total RNA in 1× CellDirect™ reaction mix, 2% SuperScript III RT platinum Taq mix and 50 nM primer mix. RT was performed at 50° C. for 15 sec and inactivated at 95° C. for 2 min. Followed are 20 thermal cycles of pre-amplification: 95° C. (15 sec) and 60° C. (4 min). Excessive primers in pre-amplification were removed by 18 units of Exonuclease I (Exo I) at 37° C. for 30 min. Pre-amplified products were diluted 1:1 with H₂O before PCR using a BioMark microfluidic instrument.

For PCR amplification, the pre-amplified products were premixed with 1× SsoFast EvaGreen supermix with low ROX (Bio-Rad, Hercules, Calif.) and 1× DNA binding dye sample loading reagent (Fluidigm). Sample and primer pre-mixtures were loaded unto 48×48 array chips according to manufacturer's protocol (cat #BMK-M-48.48, Fluidigm). Pre-amplification from about 200 pg universal mRNA and H₂O are used for positive and negative controls on each 48×48 Dynamic Array.

Single-Cell Expression Data Analysis.

Data normalization. Expression levels of 35 genes, obtained as threshold cycle (Cₜ) values, were normalized to that of the control reference gene UBB and displayed as −ΔΔCₜ values (Livak and Schmittgen, Methods 25, 402-08 (2001)). The UBB gene was used as a control because its mRNA was found to be highly stable in single prostate cells in our previous microfluidics-based PCR assays (Chen et al., Prostate 73, 813-26 (2013)). The inventors selected cells that expressed UBB at a threshold of Cₜ≦30 after pre-amplification, assuming that these cells expressing robust expression of UBB are less likely to contain degraded RNA. The −ΔΔCₜ values ranged from the lowest expression level of 0 to the highest expression level of 35, which were used to construct expression heatmaps (see FIGS. 5 and 6).

Violin plot analysis. A violin expression plot, which combines a box plot and a rotated kernel density plot (Hintze and Nelson, The American Statistician 52, 181-184 (1998)), were constructed for each gene to determine clonal distributions of gene expression in a given population of prostate cells. The density trace is plotted symmetrically to the left and the right the vertical box plot, and there is no difference in these density traces other than the direction in which they extend. Median expression levels of these genes from urinary single cells isolated in (1) normal controls and patients diagnosed with (2) benign prostate hyperplasia (BPH), (3) prostatic intraepithelial neoplasia (PIN) and (4) prostate cancer were analyzed using one-way ANOVA and unpaired Student's t test using R. A P value of <0.05 is considered as statistically significant.

Parallel coordinate plot analysis. Expression patterns of 6 genes in urinary single cells were visualized in parallel coordinate plots using the software of GGobi data visualization system (Swayne et al., Computational Statistics & Data Analysis 43, 423-444 (2003)). Each parallel coordinate plot was composed of points and lines. The points, referring to cells (total 1,220 cells), were arranged from the left to the right for each gene according to its gene expression values from the least to the highest. The lines linked to these points displayed expression connectivity among these 6 genes. Expression connectivities of selected cells for each patient were highlighted in patina color, and all the rest were in brown color (see explanations in the main text).

In silico analysis of gene expression. Gene expression (RNA-seq) data of adjacent normal (n=37) and primary PCa (n=140) used for this study were obtained from The Cancer Genome Atlas (TCGA). In order to display the expression level of selected genes in the same heat map, TCGA data were adjusted using Normalize Genes/Rows function in the software of MultipleExperiment Viewer 4.8. This process standardized gene expression values using the mean and the standard deviation of the row of the matrix to which the gene belongs. The difference between Prostate samples and Normal samples was further compared by Student's t-test using Prism 6 (GraphPad Software, La Jolla, Calif.). A P value of <0.05 is considered as statistically significant.

1. A method of detecting prostate cancer cells comprising:
(a) measuring levels of at least one biomarker in a single prostate cell isolated from post-digital rectal examination (DRE) urine of subjects; and
(b) comparing the single cell levels of the biomarker to a reference to classify the prostate cell as cancerous or non-cancerous.

2. The method of claim 1, wherein the biomarker wherein the biomarkers comprise the genes CXCL6, TGFBR2, GSK3B, CDKN1C, GATA3 and EIF4EBP1.

3. The method of claim 1, wherein a single prostate cancer cell is isolated using a dielectrophoresis cage array or micromanipulation.

4. A method of detecting prostate cancer cells in a urine sample comprising:
(a) concentrating cells in a urine sample;
(b) contacting the concentrated cells with a detectable antibody that binds a prostate specific marker; and
(c) conducting biomarker profiling on a plurality of single prostate cells.

5. The method of claim 4, wherein the prostate specific marker is prostate specific antigen (PSA) or prostate specific membrane antigen (PSMA).

6. The method of claim 5, wherein the prostate specific marker further comprises EpCAM and/or CK7/8.

7. The method of claim 4, wherein the prostate specific marker is PSA, EpCAM, and CK7/8.

8. The method of claim 4, wherein a single prostate cancer cell is isolated using a dielectrophoresis cage array.

9. A method for expressing complex gene expression patterns as binary code strings comprising: identifying and ordering a plurality biomarkers into a binary code string that is correlated with a diagnosis or prognosis, wherein the biomarkers are genes that exhibit bimodal expression in cancer.

10. The method of claim 9, wherein the biomarkers comprise the genes CXCL6, TGFBR2, GSK3B, CDKN1C, GATA3 and EIF4EBP1.

11. The method of claim 9, wherein the binary code strings are composed of a 0 representing low expression or 1 representing high expression for each gene.

12. A computer implemented method comprising the steps of (a) obtaining single cell protein level measurements of one or more biomarker, (b) transforming the obtained measurements to a score or ratio, and (c) determining if the measurements indicate the presence of prostate cancer.

13. A method of treating a patient having prostate cancer comprising: administering a treatment for prostate cancer to a patient having elevated single cell levels of one or more biomarker.

14. A method of monitoring a subject comprising:
(a) measuring levels of a biomarker in a single prostate cell isolated from post-digital rectal examination (DRE) urine of subjects periodically; and
(b) comparing the single cell levels of the biomarker to a reference to classify the prostate cell as cancerous or non-cancerous over time.

15. The method of claim 16, wherein the subject is at risk of developing prostate cancer or is undergoing prostate cancer treatment.

16. A method for determining a biomarker profile of a population of representative cells isolated from urine comprising:
(a) contacting cells isolated from urine with a detection agent that identifies a population of representative cells in the sample;
(b) isolating the identified cells as single cell isolates;
(c) conducting biomarker analysis on the each of the isolated single cells to determine a biomarker profile.

17. A method for determining a biomarker expression profile for detecting and evaluating prostate cancer in a patient comprising:
(a) contacting cells isolated from urine obtained from a patient suspected of having prostate cancer with a detection agent that identifies a population of prostate cells in the sample;
(b) isolating the identified prostate cells as single cell isolates;
(c) conducting prostate cancer biomarker analysis on the each of the isolated single cells to determine a biomarker profile;
(d) assessing the biomarker profiles of a plurality of prostate cells and providing an assessment of the patient relating to a diagnosis of prostate cancer or a prognosis for prostate cancer.

18. A method for display of a biomarker expression profile comprising:
(a) obtaining single cell biomarker profiles for a plurality of target cells isolated from a sample;
(b) grouping the single cell biomarker profiles into two or more pathological stages based on correlation of the single cell biomarker profile with a normal, benign, or pathological state;
(c) displaying geometric shapes representing various biomarker profiles, wherein the geometric shape has a size that is proportional to number of cells having a particular profile and an indicator of which state the single cell biomarker profile correlates.
